(19) United States
(12) Patent Application Publication
(10) Pub. No.: US 2014/0099694 A1
(43) Pub. Date: Apr. 10, 2014

(54) METHOD FOR OBTAINING A TISSUE-ENGINEERING PRODUCT FOR REGENERATION OF CARTILAGINOUS TISSUE

(71) Applicants: Pablo Arnau Pla Calvet, Barcelona (ES); Marta Caminal Bobet, Barcelona (ES); Joaquim Vives Armengol, Barcelona (ES); Irene Oliver Vila, Barcelona (ES); Juan Garcia Lopez, Barcelona (ES)

(72) Inventors: Pablo Arnau Pla Calvet, Barcelona (ES); Marta Caminal Bobet, Barcelona (ES); Joaquim Vives Armengol, Barcelona (ES); Irene Oliver Vila, Barcelona (ES); Juan Garcia Lopez, Barcelona (ES)

(73) Assignee: BANC DE SANG I TEIXITS, BARCELONA (ES)

(21) Appl. No.: 14/122,756

(22) PCT Filed: Apr. 30, 2012
(86) PCT No.: PCT/ES2012/070298

§ 371 Date: Nov. 27, 2013

(30) Foreign Application Priority Data
May 27, 2011 (ES) 201130871

(51) Int. Cl. A61K 35/28

(52) U.S. Cl. CPC A61K 35/28
USPC 435/174

(57) ABSTRACT

The present invention relates to a method for obtaining a tissue engineering product designed to regenerate cartilage tissue, said product comprising expanded bone marrow mesenchymal cells, a non-cellular matrix and a fibrin gel, the method comprising the steps of: (a) expanding the mesenchymal cells; (b) conjugating the mesenchymal cells to the matrix; (c) washing the product obtained in step (b); and (d) mixing the product obtained in step (c) with a fibrin gel.

### CROSS REFERENCE TO RELATED APPLICATIONS

This application is a 371 application of International Application No. PCT/ES2012/070298, filed Apr. 30, 2012, which claims priority to Spanish Patent Application No. ES201130871 filed May 27, 2011, each of which is incorporated by reference in its entirety herein.

### DESCRIPTION

The present invention relates to a method for preparing a tissue engineering product designed to regenerate articular cartilage tissue. More particularly, the present invention relates to a method for preparing a product which mainly comprises expanded mesenchymal cells obtained from bone, which are expanded, immobilised on scaffolds, copolymerised and held in the implantation site by means of fibrin gels and/or mechanical insertion. The method of the present invention may further comprise a freezing stage of the tissue engineering product obtained, such that reserve copies of said product are available for safety purposes.

Due to the limited ability of articular cartilage to regenerate, the defects caused by traumas or degenerative diseases constitute an unresolved clinical problem. In pathologies of this type, the appearance of lesions is associated with pain, loss of mobility and progressive incapacity relating to the onset of arthritis or arthrosis, all of which seriously impact the quality of life of those affected.

Lesions of this type are most commonly treated with mosaicplasty, autologous chondrocyte transplantation, heterologous transplantation of osteochondral tissue, and abrasion arthroplasty or drilling. In all of these techniques, specialised cells are supplied to the affected area in order to stimulate the regeneration/replacement of the damaged tissue.

Abrasion arthroplasty or drilling is among the treatments with the highest success rates. This procedure is based on causing, by bleeding, bone marrow mesenchymal cells to flood from the subchondral bone towards the affected area such that they repair the lesion (Mitchell N et al., The resurfacing of adult rabbit articular cartilage by multiple perforations through the subchondral bone, J Bone Joint Surg Am. 1976; 58(2): 230-3.24, Levy A. et al. Chondral delamination of the knee in soccer players, AM J Sports Med. 1996; 24). The success of this technique relies on the correct integration, proliferation and differentiation of the mesenchymal cells in the area to be repaired. Owing to the inherent high level of variability in the technique, the results of treatments of this type are difficult to predict. Consequently, the lesion is often repaired with fibrocartilage instead of hyaline cartilage, which limits the quality and durability of the therapy (Pelmari K et al. Do we really need cartilage tissue engineering! Swiss Med WKLY 2009; 139: 41-42; Kreuz P C et al., Results after microfracture of full-thickness chondral defects in different compartments in the knee. Osteoarthritis. Cartilage. 2006; 14(11): 1119-25; Temenoff J S et al. Review: tissue engineering for regeneration of articular cartilage, Biomaterials. 2000;431-40)

Therefore, new technologies which solve the issues of the previous technique need to be developed. The present invention discloses a new alternative therapy based on tissue engineering. This procedure is based on using a product which comprises a synthetic-material- or biomaterial-based matrix combined with cells and hydrogels which stimulates the regeneration of damaged cartilage tissue.

The matrices used in the present invention must have special characteristics which allow them to carry out their function as a scaffold for reconstructing the target tissue, i.e. they must be biocompatible, have suitable mechanical properties in relation to the implantation site, have structural integrity and they must be bioresorbable. Moreover, these matrices must be capable of creating a biological environment which ensures that nutrients are delivered to the cells so as to ensure that said cells can carry out their regenerative function. In addition to the aforementioned characteristics, a matrix designed to regenerate cartilage tissue should also be capable of stimulating the differentiation of stem cells into chondrocytes, which should then in turn be stimulated in said matrix to form cartilage tissue.

The cells, which constitute the other component of the tissue engineering product of the present inventions, should also exhibit special characteristics such as being easily and reliably obtained, coming from a reliable source in cytogenetic terms, and having multipotent properties, which allow said cells to differentiate into chondrocytes, the cells responsible for producing cartilage tissue.

Therefore, the present invention discloses a method for preparing a tissue engineering product designed to regenerate cartilage. Said method is based on obtaining and using autologous, expanded bone marrow mesenchymal cells which are expanded and immobilised on synthetic-polymer- or biomaterial-based scaffolds. The obtained product as a whole is then implanted and fixed in the area of the lesion by means of fibrin gels and/or mechanical insertion.

One of the advantages with regard to autograft treatments, such as chondroplasty or subchondral bleeding, is that, by using the product prepared according to the method of the present invention, the patient receiving the therapy does not have to undergo one or more arthroscopic surgical procedures in order to extract the biological material necessary for regenerating the damaged joint. The cellular material required is extracted from bone marrow, which can be done on an outpatient basis. Therefore, this procedure avoids the potential complications associated with obtaining autologous chondral material, such as pain, bleeding, discomfort or complications due to infection.

Another advantage in relation to the technique of abrasion arthroplasty or drilling used in cases of small lesions, which technique consists in damaging the subchondral bone so as to enable the supply of regenerative cells, is that, by using the product prepared according to the method of the present invention, it is possible to administer a known and perfectly defined dose of cells, which makes it possible to achieve a reproducible treatment for patients undergoing this therapy.

An additional advantage is that it is possible to implant a product which is perfectly adapted to the topology of the lesion.

In this aspect, the present invention discloses a method by means of which the matrices are conjugated to a cellular product rich in mesenchymal cells by means of an immobilisation process. Said cellular product having the recognised ability to stimulate chondrocytes is obtained by selecting and subsequently expanding said cells in vitro. By manipulating the expansion process of the cells, the method of the present invention makes it possible to create many different amounts of cartilage graft, which facilitates the treatment of lesions of different sizes and origins, thus making it applicable across a wide spectrum of therapeutic applications aimed at restoring damaged cartilage.

Another characteristic of the method of the present invention is that the cells are grown using human growth medium supplements, which enables rapid expansion of said cells and prevents possible adverse effects arising from the cells coming into contact with non-human (or humanised) components. One advantage of the method of the present invention is that, once the expansion is complete, a part of the cells is cryogenically preserved such that doses are retained for use in future treatments.

Moreover, conjugating the mesenchymal cells to the biomatrices by means of a colonisation process allows the chondrogenic cells to be located in the area to be regenerated. This characteristic, which differs from other applications such as abrasion arthroplasty or drilling, ensures the reparative action of the cells in the area to be treated and, in contrast to other tissue engineering procedures, guarantees that the stem cells remain in the area of the lesion where they are to carry out the repair work. Finally, combining these particles with mesenchymal cells by means of fibrin gels results in an end product having plasticity and being easy-to-handle, in turn enabling the mixture to be immobilised in the area to be treated and thus avoiding structural incoherence with the environment to be regenerated.

The present invention discloses a method for obtaining a tissue engineering product designed to regenerate cartilage tissue, said product comprising expanded bone marrow mesenchymal cells, a non-cellular matrix and a hydrogel-based component, said method comprising the steps of:

(a) expanding the mesenchymal cells:

(b) conjugating the mesenchymal cells to the matrix;

(c) washing the product obtained in step (b); and

(d) mixing with a fibrin gel.

A person skilled in the art knows how to expand the mesenchymal cells so as to obtain the number of cells required for conjugating to the matrix; this number will depend on the size of the lesion to be treated. Once said required number has been obtained, the conjugation stage of said cells to the matrix is initiated. This conjugation can be carried out using agitated or non-agitated systems.

The mesenchymal cells obtained in the expansion stage are resuspended in DMEM growth medium supplemented with serum to a concentration of between 1×103 to 1×107 cells per millilitre. If necessary, said cells can be preserved cryogenically for future use.

Next, the cell suspension is dispensed in a sterile manner into a cell bag or culture flask which has been loaded with the matrix beforehand and which can be provided with a pendular shaker, in order to conjugate the mesenchymal cells to the matrix. The ratio of the number of cells per cubic centimetre is between 1×102 to 1×108 cells per cubic centimetre of matrix. The mixture is then left to incubate for between 1 to 24 hours at a temperature of 35-38° C., with CO2 saturation levels of 2.5-7.5% and relative humidity above 90%. IF the mixture is to be agitated, the agitation conditions used in order to ensure anchoring of the cells are 1 to 120 revolutions per minute.

The suspension of mesenchymal cells immobilised on the matrix, which was obtained in step (b), is washed a plurality of times using a physiological saline solution and is dispensed in a sterile manner into a storage bag.

In step (d) of the method of the present invention, the product obtained in step (c) is mixed in a sterile manner with a fibrin gel in a ratio of 0.1 to 10 units of volume of fibrinogen solution for each 0.1-10 units of volume of thrombin solution and 0.1-10 units of volume of colonised matrix obtained in step (c).

The present invention will be described in more detail below with reference to examples. These examples, however, should not be considered limiting to the technical scope of the present invention.

### EXAMPLES

### Example 1

Method for preparing one cubic centimetre of the tissue engineering product designed to regenerate cartilage according to the present invention.

12×106 bone marrow mesenchymal cells were obtained by means of expansion using a growth medium free of animal serum and were inoculated into a culture flask. A DMEM-based medium supplemented with human serum to a ratio of 10% (v/v) was used. The concentration of cells was set at 6×105 cells per millilitre. The cell suspension was added in a sterile manner to a second culture flask made of plastics material and provided with a pendular shaker, into which culture flask 1 cubic centimetre of matrix had been added beforehand. The remaining 6×106 cells were preserved cryogenically so as to allow further treatments to be carried out if necessary.

The mixture was incubated under the following conditions: at a temperature of 37° C. and with CO2 saturation levels of 5% and relative humidity of 95%. The agitation cycle was then initiated and carried out for 24 hours at a speed of between 120-200 rpm. Afterwards, the product obtained was washed such that cellular residues and the growth medium were removed. This was carried out using a physiological saline solution and the product obtained was packed in a sterile bag.

Just prior to administering the product to the patient, the matrices are combined with a fibrin gel, which process is carried out by using a ratio by volume consisting of one unit of fibrinogen solution to one unit of volume of thrombin and one unit of volume of the osseous colonised matrix. Once combined, the product is given the form that allows it to adapt to the spatial distribution of the lesions and it is placed on said lesion.

Although the invention has been described with reference to a preferred embodiment, this should not be considered limiting to the present invention which will be defined by the wider interpretation in the following claims.

What is claimed is:

1. Method for obtaining a tissue engineering product designed to regenerate cartilage tissue, said product comprising expanded bone marrow mesenchymal cells, a non-cellular matrix and a fibrin gel, comprising the steps of:
(a) expanding the mesenchymal cells;
(b) conjugating the mesenchymal cells to the matrix;
(c) washing the product obtained in step (b); and
(d) mixing the product obtained in step (c) with a fibrin gel;
characterised in that said matrix is a biomaterial.

2. Method according to claim 1, characterised in that step (b) is carried out using an agitated or non-agitated system.

3. Method according to claim 1, characterised in that it further comprises a cryopreservation step of the expanded mesenchymal cells for future use thereof.

4. Method according to claim 1, characterised in that the ratio of the number of cells per cubic centimetre of matrix in step (b) is within the range of between 1×10² and 1×10³ cells per cubic centimetre of matrix.

5. Method according to claim 1, characterised in that step (b) is carried out for 1 to 24 hours, at a temperature of 35-38° C. and with CO₂ saturation levels of 2.5-7.5% and relative humidity of over 90%.

6. Method according to claim 1, characterised in that the product is washed in step (c) a plurality of times using a physiological saline solution.

7. Method according to claim 1, characterised in that the mixing with a fibrin gel in step (d) is carried out with a ratio of 0.1 to 10 units of volume of fibrinogen solution to each 0.1-10 units of volume of thrombin solution and 0.1-10 units of volume of colonised matrix obtained in step (c).
